(19) United States
(12) Patent Application Publication
(10) Pub. No.: US 2019/0142738 A1
(43) Pub. Date: May 16, 2019

(54) TRANSDERMAL FORMULATIONS FOR DELIVERY OF CELECOXIB AND ITS USE IN THE TREATMENT OF CELECOXIB-RESPONSIVE DISEASES AND CONDITIONS

(71) Applicant: DELIVRA INC., Burlington (CA)

(72) Inventors: Joseph Gabriele, Stoney Creek (CA); Mikaela Teris, Montreal (CA)

(21) Appl. No.: 16/300,116

(22) PCT Filed: May 16, 2017
(86) PCT No.: PCT/CA2017/050585

§ 371 Date: Nov. 9, 2018

Related U.S. Application Data
(60) Provisional application No. 62/336,962, filed on May 16, 2016.

(51) Int. Cl. A61K 9/00; A61K 31/635; A61K 9/107; A61K 47/14; A61K 47/44; A61K 47/30; A61K 47/46; A61K 47/24; A61K 9/06; A61K 47/22

(52) U.S. Cl. CPC A61K 9/0014; A61K 31/635; A61K 9/107; A61K 47/14; A61K 47/22; A61K 47/30; A61K 47/46; A61K 47/24; A61K 9/06; A61K 47/44

(57) ABSTRACT

The present application is directed to transdermal formulations for the delivery of celecoxib to a subject for the treatment of celecoxib-responsive diseases or conditions. In particular, the transdermal formulation is an emulsion comprising an oil phase, an aqueous phase and an external phase.

### CROSS-REFERENCE TO RELATED APPLICATIONS

This application claims the benefit of U.S. Provisional Patent Application Ser. No. 62/336,962, filed May 16, 2016, the content of which is hereby incorporated by reference in its entirety.

### FIELD

The present application relates to transdermal formulations for effective delivery of celecoxib and various methods of use thereof.

### BACKGROUND

Celecoxib (Celebrex®) is the first nonsteroidal anti-inflammatory drug (NSAID) which is a selective cyclo-oxygenase (COX) 2 inhibitor (coxib) to be introduced into clinical practice. Celecoxib remains an effective and useful alternative to nonselective NSAIDs in the treatment of acute or chronic pain. As a selective COX-2 inhibitor, celecoxib offers the prospect of improved gastrointestinal (GI) tolerability in comparison to nonselective COX isoenzyme (COX-1/COX-2) inhibitors (Keating et al., 2007).

Currently celecoxib has been approved for the relief of the signs and symptoms of osteoarthritis (OA) and rheumatoid arthritis (RA), juvenile RA, ankylosing spondylitis, management of acute pain in adults, treatment of primary dysmenorrhea and for the reduction in the number of adenomatous colorectal polyps in familial adenomatous polyposis (Keating et al., 2007).

The premature metabolism of drugs as a result of the first-pass effect has made transdermal delivery an attractive and alternative strategy (Prausnitz, et al. 2008). Topical administration circumvents issues of low bioavailability and additionally results in fewer adverse effects and lower systemic drug concentration.

For many years, people have placed natural substances on the skin for local ailments. However, the human skin acts as a formidable barrier due in large part to the stratum corneum, which mostly consists of a lipid-enriched matrix and blocks entry of most topically applied agents, with the exception of low molecular weight, lipid-soluble drugs. This poses a challenge for administrating medications via the skin for either local cutaneous or systemic therapy.

Transdermal drug delivery strategies have thus focused primarily on the manipulation of the lipid milieu of the skin. In particular, penetration enhancers which interact with skin constituents to promote drug transport have provided an approach to increase the range of therapeutic agents that can be delivered transdermally.

Despite the significant permeability barrier of the stratum corneum, drug delivery via the skin is a very attractive option and is widely employed for both local and systemic therapy. Topical treatment of cutaneous disorders obviously targets the site of disease, thereby minimizing adverse side effects elsewhere within the body. Delivery of systemic therapies via the skin avoids degradation of the medication within the gastrointestinal tract and first-pass metabolism by the liver, both of which are associated with oral administration of drugs, in addition to evading the pain and safety issues associated with injections. Transdermal delivery of drugs, in some cases, enables infrequent dosing and maintenance of steady state drug levels.

Therefore, it is desirable to provide improved clinical applications of celecoxib, through transdermal therapeutic compositions and delivery systems.

### SUMMARY

The present application includes transdermal formulations for the delivery of celecoxib to a subject. In some embodiments, the formulation comprises at least three phases including at least one oil phase, at least one aqueous phase and at least one external phase comprising celecoxib.

In some embodiments, the present application includes a transdermal formulation comprising:
(a) an aqueous phase comprising water and at least one water soluble emulsion stabilizer;
(b) an oil phase comprising at least one emulsifier, at least one oil soluble emulsion stabilizer, at least one emollient comprising at least one flavonoid and at least one other emollient;
wherein the oil and aqueous phase form an emulsion;
(c) an external phase comprising at least one phospholipid-complexed flavonoid and celecoxib; and
(d) at least one preservative phase.

The present application also includes methods for treating one or more celecoxib-responsive diseases and conditions comprising administering an effective amount of one or more of the transdermal formulations of the application to a subject in need thereof. In some embodiments, the celecoxib-responsive disease or condition is selected from one or more of acute pain, chronic pain, nociceptive pain, neuropathic pain, inflammation and cancer.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows the stability of exemplary formulation 1 over 3 months at 45° C. for pH and viscosity evolution.

FIG. 2 shows the stability of exemplary formulation 2 over 3 months at 45° C. for pH and viscosity evolution.

FIG. 3 shows the stability of exemplary formulation 3 over 3 months at 45° C. for pH and viscosity evolution.

FIG. 4 shows the stability of exemplary formulation 4 over 3 months at 45° C. for pH and viscosity evolution.

FIG. 5 shows the celecoxib concentration in exemplary formulation 4 in serum.

FIG. 6 shows the celecoxib concentration in exemplary formulation 4 in synovial fluid.

FIG. 7 shows the cumulative pain scores for Celebrex product treatment groups at each assessment time point in comparison to no treatment, exemplary transdermal formulation 1, Voltaren and positive control meloxicam.

FIG. 8 shows the cumulative pain scores for exemplary transdermal formulation 1 comprising Celebrex at each assessment time point in comparison to no treatment and Voltaren.

FIG. 9 shows the mean percent baseline knee cap measurements for Celebrex product treatment groups at each assessment time point in comparison to no treatment, transdermal base formulation 1, Voltaren and positive control oral meloxicam.

FIG. 10 shows the mean percent baseline knee cap measurement for transdermal Celebrex in exemplary formulation 1 at each assessment time point in comparison to no treatment and Voltaren.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The present description refers to a number of chemical terms and abbreviations used by those skilled in the art. Nevertheless, definitions of selected terms are provided for clarity and consistency.

As used in this application, the singular forms “a”, “an” and “the” include plural references unless the content clearly dictates otherwise. For example, an embodiment including “an agent” should be understood to present certain aspects with one compound or two or more additional compounds.

In embodiments comprising an “additional” or “second” component, such as an additional or second agent, the second component as used herein is chemically different from the other components or first component. A “third” component is different from the other, first, and second components, and further enumerated or “additional” components are similarly different.

The term “agent” as used herein indicates a compound or mixture of compounds that, when added to a formulation, tend to produce a particular effect on the formulation's properties.

The term “thickening agent” as used herein refers to a compound or mixture of compounds that adjusts the thickness of the formulation.

The term “and/or” as used herein means that the listed items are present, or used, individually or in combination. In effect, this term means that “at least one of” or “one or more” of the listed items is used or present.

The term “suitable” as used herein means that the selection of the particular compound or conditions would depend on the specific synthetic manipulation to be performed, and the identity of the molecule(s) to be transformed, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The term “water soluble”, for example as in “water soluble emulsion stabilizer”, refers to a substance that has a solubility in aqueous based solutions that is sufficient for the substance to exert its desired effect at concentrations that are pharmaceutically acceptable.

The term “oil soluble”, for example as in “oil soluble emulsion stabilizer”, refers to a substance that has a solubility in oil based solutions that is sufficient for the substance to exert its desired effect at concentrations that are pharmaceutically acceptable.

“Formulation” and “pharmaceutical formulation” as used herein are equivalent terms referring to a formulation for pharmaceutical use.

The term “pharmaceutically acceptable” means compatible with the treatment of animals, in particular, humans.

The term “effective amount” as used herein means an amount sufficient to achieve the desired result and accordingly will depend on the ingredient and its desired result. Nonetheless, once the desired effect is known, determining the effective amount is within the skill of a person skilled in the art.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e. not worsening) the state of disease, prevention of disease spread, delaying or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. Treatment methods comprise administering to a subject a therapeutically effective amount of an active agent and optionally consists of a single administration, or alternatively comprises a series of applications. The length of the treatment period depends on a variety of factors, such as the severity of the condition, the age of the patient, the concentration of active ingredient or agent, the activity of the compositions described herein, and/or a combination thereof. It will also be appreciated that the effective dosage of the agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for duration sufficient to treat the patient.

“Topical composition” as used herein includes a composition that is suitable for topical application to the skin, nail, mucosa, wound bed or wound cavity. A topical composition may, for example, be used to confer a therapeutic or cosmetic benefit to its user. Specific topical compositions can be used for local, regional, or transdermal application of substances.

The term “topical administration” is used herein to include the delivery of a substance, such as a therapeutically active agent, to the skin or a localized region of the body.

“Transdermal” as used herein includes a process that occurs through the skin. The terms “transdermal,” “percutaneous” and “transcutaneous” can be used interchangeably. In certain embodiments, “transdermal” also includes epicutaneous. Transdermal administration is often applied where systemic delivery of an active is desired, although it may also be useful for delivering an active to tissues underlying the skin with minimal systemic absorption.

“Transdermal application” as used herein includes administration through the skin. Transdermal application can be used for systemic delivery of an active agent; however, it is also useful for delivery of an active agent to tissues underlying the skin with minimal systemic absorption. In certain embodiments, “transdermal application” can also include epicutaneous application.

The term “emollient” as used herein refers to a compound or mixture of compounds that adds or replaces natural oils in the skin, for example by maintaining the integrity of the hydrolipids of the skin.

The term “polar emollient” as used herein refers to emollient compounds, which are generally oils, having heteroatoms that differ in electronegativity. This results in a dipole moment. Typical polar oils are fatty alcohols, esters and triglycerides. While they are still water insoluble and oil-loving, these oils have unique characteristics due to their polar nature. They typically combine with higher hydrophobic lipid balance (HLB) emulsifiers to make stable emulsions, they dissolve materials that are insoluble in nonpolar oils, and they provide unique properties when compared with nonpolar oils such as mineral oil.

The term “medium polar emollient” as used herein refers to emollient compounds, which are generally oils that are less polar than the polar emollients but still more polar than nonpolar oils such as mineral oil.

The term “humectant” as used herein refers to a compound or mixture of compounds intended to increase the water content of the top layers of skin.

The term “emulsifier” of “emulsifying agent” as used herein refers to a compound of mixture of compounds which promote or facilitate the dispersion of one substance in another to form an emulsion.

The term “penetration enhancer” as used herein refers to a compound or mixture of compounds that improves the rate of percutaneous transport of an active agent across the skin for use and delivery of active agents to organisms such as mammals.

The term “preservative” as used herein refers to a substance that is added to products such as pharmaceutical compositions, to prevent decomposition by microbial growth or by undesirable chemical changes. For example, the addition of antimicrobial preservatives prevents microorganism growth by modifying the pH level.

The term “flavonoid compounds” as used herein refers to a class of plant secondary metabolites that have the general structure of a 15-carbon skeleton, which contains two phenyl rings (A and B) and heterocyclic ring (C). The basic chemical structure of a flavonoid as used herein is as follows:

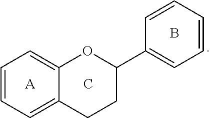

However, the term flavonoid includes the following flavonoids:

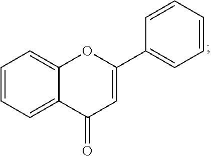 [SMILES: O=c1cc(-c2ccccc2)oc2ccccc12]

isoflavonoids:

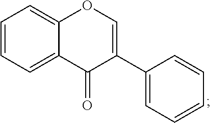 [SMILES: O=c1c(-c2ccccc2)coc2ccccc12]

and neoflavonoids:

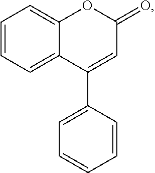 [SMILES: O=c1cc(-c2ccccc2)c2ccccc2o1]

as well as their non-ketone containing counterparts, known as flavanoids. Flavonoids are one of the largest known nutrient families, and include over 6,000 already-identified family members. Some of the best-known flavonoids include rutin, quercetin, kaempferol, catechins, and anthocyanidins. This nutrient group is most famous for its antioxidant and anti-inflammatory health benefits, as well as its contribution of vibrant color to foods.

The term “celecoxib” as used herein refers to a COX-2 selective nonsteroidal anti-inflammatory drugs (NSAID). Celecoxib has the following structure:

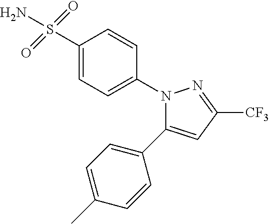 [SMILES: Cc1ccc(-c2cc(C(F)(F)F)nn2-c2ccc(S(N)(=O)=O)cc2)cc1]

and includes tautomers thereof and pharmaceutically acceptable salts and solvates thereof.

The term “pharmaceutically acceptable salt” means an acid addition salt or basic addition salt which is suitable for or compatible with the treatment of subjects, including human subjects.

The term “pharmaceutically acceptable acid addition salt” as used herein means a compound formed by the reaction of a pharmaceutically acceptable acid with a basic compound. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Such salts may exist in either a hydrated, solvated or substantially anhydrous form. In general, the acid addition salts are more soluble in water and various hydrophilic organic solvents, and generally demonstrate higher melting points in comparison to their free base forms. The selection of the appropriate salt will be known to one skilled in the art. Other non-pharmaceutically acceptable acid addition salts, e.g. oxalates, may be used, for example, in the isolation of the compounds of the invention, for laboratory use, or for subsequent conversion to a pharmaceutically acceptable acid addition salt.

The term “pharmaceutically acceptable basic addition salt” as used herein means any pharmaceutically acceptable organic or inorganic base addition salt of any acid compound. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art. Other non-pharmaceutically acceptable basic addition salts, may be used, for example, in the isolation of the compounds for laboratory use, or for subsequent conversion to a pharmaceutically acceptable basic addition salt.

The term “wt %” means a percentage expressed in terms of weight of the ingredient or agent over the total weight of the formulation multiplied by 100.

The term “w/w” as used herein means the number of grams of solute in 100 g of solution.

The term “water” as used herein as an ingredient in the formulations of the application refers to pharmaceutically acceptable water.

### II. Formulations of the Application

In some embodiments, the transdermal formulation base of the present application comprises:
(a) an aqueous phase comprising water and at least one water soluble emulsion stabilizer;
(b) an oil phase comprising at least one emulsifier, at least one oil soluble emulsion stabilizer, at least one emollient comprising at least one flavonoid and at least one other emollient;
wherein the oil and aqueous phase form an emulsion;
(c) an external phase comprising at least one phospholipid-complexed flavonoid and celecoxib; and optionally
(d) at least one preservative phase

In some embodiments, the transdermal formulation base comprises an oil-in-water emulsion. In some embodiments, the formulation is a multiphase emulsion, such as an oil-in-water-oil emulsion or a water-in-oil-water emulsion.

### Emulsifiers

In some embodiments the emulsifier is any oil-soluble fatty acid ester or mixture of fatty acid esters in which the fatty acid esters have a fatty acid composition similar to the fatty acid composition of skin for generating skin-compatible liquid crystals and to mimic the molecular organization of the intracellular lipidic laminae of the stratum corneum. Such liquid crystals are able to rapidly cross skin layers as well as to integrate into the skin's own lipid barrier to provide strength and greater integrity to this barrier.

In some embodiments the fatty acid esters are selected from sugar alcohol and fatty acid alcohol esters of any C₁₄-C₂₆-fatty acid or mixtures thereof. In some embodiments, the fatty acid esters are esters of fatty acids that are present in olive oil, palm oil and/or canola oil. In some embodiments, the fatty acids are esterified with fatty acid alcohols such as, but not limited to, cetyl alcohol, cetaryl alcohol, lauryl alcohol, stearyl alcohol, myristyl alcohol and/or oleyl alcohol. In some embodiments, the fatty acids are esterified with sugar alcohols such as, but not limited to, sorbitol, glycerol, mannitol, inositol, xylitol, erythritol, threitol, arabitol and/or ribitol. Olive oil fatty acid esters, and their use in transdermal formulations is described, for example, in U.S. Patent Application Publication No. 2011/0021439.

In some embodiments, the emulsifier is present in the formulations of the application in an amount of about 1 wt % to about 10 wt %, about 2 wt % to about 9 wt %, or about 3 wt % to about 5 wt %.

In some embodiments, an emulsifier is optionally included in the external phase. In some embodiments, the emulsifier in the external phase is glycerin or a glycerin/soy bean extract mixture.

In some embodiments, the emulsifier in the external phase is present in the formulations of the application in an amount of about 0.5 wt % to about 2 wt %.

### Emulsion Stabilizers

In some embodiments, the emulsion stabilizer is any compound or mixture of compounds that helps to maintain the oil-in-water emulsion. There are three types of emulsion instability: flocculation, coalescence and creaming. Flocculation describes the process by which the dispersed phase comes out of suspension in flakes. Coalescence is another form of instability, which describes when small droplets combine to form progressively larger ones. Emulsions can also undergo creaming, which is the migration of one of the substances to the top or bottom (depending on the relative densities of the two phases) of the emulsion under the influence of buoyancy or centripetal force when a centrifuge is used. Generally, emulsion stability refers to the ability of an emulsion to resist change in its properties over time. In the present application an emulsion stabilizer is present in both the oil phase and the aqueous phase.

In some embodiments, the oil soluble emulsion stabilizer is one or more waxes. In some embodiments the waxes are selected from animal and plant waxes and mixtures thereof. In some embodiments, the plant wax is a wax derived from berries, olives or from palm (e.g. carnauba wax). The one or more waxes are stabilizers that are present in the oil phase of the formulation.

In some embodiments, the oil soluble emulsion stabilizer is present in the formulation in an amount of about 0.5 wt % to about 4 wt % or about 1 wt % to about 3 wt %.

In some embodiments, the water soluble emulsion stabilizer is one or more thickening agents. In some embodiments, the thickening agents are any compound or mixture of compounds that maintains components in the formulation in suspension and provides a suitable consistency to the formulation.

In some embodiments, the water soluble emulsion stabilizer is selected from natural polymers, gums and synthetic polymers, and mixtures thereof. In some embodiments, natural polymers, gums and synthetic polymers, and mixtures thereof, are water soluble and therefore are present in the aqueous phase of the formulation. In some embodiments, the natural polymers are selected from alginic acid and derivatives thereof, cellulose and derivatives thereof and scleroglucans, and mixtures thereof. In some embodiments, the gums are selected from xanthan gum, tara gum, guar gum and arabic gum, and mixtures thereof. In some embodiments, the synthetic polymers are selected from polyacrylates, polyisobutenes and polysorbates, and mixtures thereof.

In some embodiments, the water soluble emulsion stabilizer is present in the formulations of the application in an amount of about 0.01 wt % to about 2 wt %, about 0.05 wt % to about 1.5 wt %, or about 0.1 wt % to about 1 wt %.

### Emollient Comprising at Least One Flavonoid

In some embodiments, the one or more emollients comprising one or more flavonoid compounds are polar emollients. Polar emollients generally include natural oils and extracts from plants. In some embodiments, the polar emollients are derived from fruits (including berries), vegetables, herbs, spices, legumes, leaves, seeds and/or grains. In some embodiments, the polar emollient is a natural oil or extract from citrus, Ginkgo biloba, tea, wine, cacao, onion, kale, parsley, red beans, broccoli, endive, celery, cranberries, blackberries, red raspberries, blackcurrants, acai, blueberries, bilberries, milk thistle, apples, hawthorn, Echinacea, grapes, and/or soy.

In some embodiments, the polar emollient comprising one or more flavonoid compounds is a natural oil or extract from the genera Rubus, Ribes, Argania, Nymphaea, Peucedanum or Imperatoria, Sambucus, Calendula, Butea, Citrus (e.g. lime), or species or subspecies thereof. In some embodiments, the polar emollient comprising one or more flavonoid compounds comprises Leptospermum Scoparium and/or manuka oil. In some embodiments, the polar emollient comprising one or more flavonoid compounds comprises Argan oil, Cicatrol, Protectol, and/or Calendula.

In some embodiments, the emollients comprising one or more flavonoid compounds are present in the formulations of the application in an amount of about 1 wt % to about 20 wt %, about 1.5 wt % to about 15 wt %, or about 2 wt % to about 10 wt %.

### Further Emollients

The polarity of the emollients used in the present can vary depending on the identity of the emulsifiers and emulsion stabilizers, however can nonetheless be selected by a person skilled in the art. In some embodiments, the formulations of the present application comprise both polar emollients and medium polar emollients.

In some embodiments, further polar emollients used in the present application comprise oil from an animal in the family Dromaius, for example Dromiceius (emu) or a plant, such as, Jojoba oil, Olive oil and/or coconut oil.

In some embodiments the one or more further polar emollients are present in an amount of about 0.5 wt % to about 15 wt %, about 1 wt % to about 10 wt %, or about 2 wt % to about 8 wt %.

In some embodiments, the medium polar emollient is an ester such as octyl palmitate, isopropyl stearate and isopropyl palmitate, or an alcohol such as octyl dodecanol, or mixtures thereof.

In some embodiments the emollients also act as a thickener (stabilizer) and/or a humectant.

In some embodiments, the one or more medium polar emollients are present in an amount of 0.5 wt % to about 10 wt %, about 1 wt % to about 7 wt %, or about 1.5 wt % to about 5 wt %.

### Flavonoid-Containing Extract

In some embodiments, the one or more flavonoid-containing extracts water phase is any suitable water soluble natural extract comprising a flavonoid with anti-inflammatory and/or antioxidant properties. In some embodiments, the one or more flavonoid-containing extracts are plant-based extracts, including but not limited to, one or more of Nymphaea caerulea flower extract, Peucedanum ostruthium leaf extract, Sambuscus nigra extract, Calendula flower Extract, Gingko biloba extract, Imperatoria Alpaflor extract, Sambucus Alpaflor extract, Blue lotus extract, Calendula Alpaflor extract, Masterwort extract, Elderberry extract, Angelica extract, green tea extract, chamomile extract, pomegranate pericarp and Peucedanum ostruthium leaf extract.

In some embodiments, the one or more flavonoid-containing extracts for the external phase are present in an amount of about 0.1 wt % to about 15 wt %, about 0.5 wt % to about 10 wt %, or about 1 wt % to about 8 wt %.

### Phospholipid-Complexed Flavonoid

In some embodiments, the flavonoid in the phospholipid-complexed flavonoid is a bioflavonoid isolated from plants such as, but not limited to, Gingko bilboa, Crataegus sp., Passiflora incarnata, Tormentilla potentilla, Tea sinensis., Aurantium sp., Citrus sp., Eucaliptus sp., Matricaria chamomilla, Rheum sp. and Fagara sylanthoides. In some embodiments, the flavonoid is isolated from green tea, buckwheat, the leaves and petioles of asparagus, fruit of the Fava D-Ante tree, fruits and fruit rinds, for example from citrus fruits such as orange, grapefruit, lemon and lime, and berries such as mulberries and cranberries. In some embodiments, the flavonoid is selected from quercetin, myrcetin, apigenin and rutin, and mixtures thereof.

In some embodiments, the phospholipid is any phospholipid, or mixture of phospholipids, from a plant or animal, or any synthetic phospholipid. In some embodiments, the phospholipid is selected from a phosphatidylcholine, a phosphatidylethanolamine, a phosphatidylinostinol, a phosphatidylserine and lecithin, and mixtures thereof.

In some embodiments, the phospholipid-complexed flavonoid is commercially available. In some embodiments, the phospholipid-complexed flavonoid is prepared by combining the phospholipid and flavonoid in a suitable solvent or mixture of solvents, in a mole ratio of phospholipid:flavonoid of about 0.5 to 2, or about 1, and isolating the resulting complex, for example, but removal of the solvent(s), precipitation and/or lyophilization.

In some embodiments, the phospholipid-complexed flavonoid of the external phase is present in an amount of about 0.5% wt % to about 5 wt %, about 1 wt % to about 4 wt %, or about 1.5 wt % to about 2.5 wt %.

Complexes of bioflavonoids with phospholipids, their preparation and use, are described, for example in U.S. Pat. No. 5,043,323, the contents of which are incorporated by reference in their entirety.

### Celecoxib

In an embodiment of the application, the transdermal formulations comprise celecoxib.

In some embodiments, celecoxib is present in the formulation in an amount of about 0.1 wt % to about 15 wt %, about 1 wt % to about 10 wt % or about 2 wt % to about 8 wt %.

In some embodiments, celecoxib is in the external phase.

### Water

The balance of the aqueous phase of the composition is made up of water. Further, it is an embodiment that the solvent for the external phase and/or the preservative phase (if present) comprises water. In some embodiments, the water is purified and/or demineralized water. The purified water may, for example, be filtered or sterilized.

In some embodiments, the amount of water in the aqueous phase is about 30 wt % to about 70 wt %, or about 40 wt % to about 65 wt % (based on the total weight of the formulation).

In some embodiments, the amount of water in the external phase is about 1 wt % to about 20 wt %, or about 3 wt % to about 10 wt % (based on the total weight of the formulation).

### Preservatives

In some embodiments, the formulations of the present application comprise at least one preservative. Preservatives include antimicrobial agents. In some embodiments the preservatives prevent or inhibit the growth of micro-organisms, including bacteria, yeasts and molds. In some embodiments, the preservatives prevent or inhibit undersirable chemical reactions from occurring. For example, in some embodiments, the preservative is an antioxidant.

In some embodiments, the preservative comprises a preservative system comprising phenoxyethanol, benzoic acid, and dehydroacetic acid. In some embodiments, the preservative comprises capryl glycol, which also advantageously has humectant and emollient properties. In some embodiments, the preservative comprises chlorphensin. In some embodiments, the preservative comprises ethylhexylglycerin which also advantageously has skin conditioning and emollient properties and acts as a deodorant. In some embodiments, the preservative comprises a natural antimicrobial agent (antibacterial, antifungal, antiviral). In some embodiments, the natural antimicrobial agent is selected from tea tree oil (Malaleuca alternifolia leaf oil) and myrtyl lemon essential oil. In some embodiments, the preservative comprises a preservative and a preservative booster.

In some embodiments, other components of the formulation have intrinsic anti-microbial properties.

In some embodiments, the one or more preservatives are present in an amount of about 0.01 wt % to about 5 wt %, about 0.05 wt % to about 4 wt %, or about 0.1 wt % to about 2 wt %.

### Further Optional Ingredients

In some embodiments, the formulations of the present application further comprise additional ingredients that are common in the transdermal base formulation art. These ingredients are, for example, but not limited to, further active pharmaceutical ingredients, pH adjusters or buffering agents, further solvents, solubilizers, chelating agents, pigments, fragrances, humectants, solubilizers, penetration enhancers, antioxidants and/or reducing agents.

(a) pH Adjusters/Buffering Agents

In some embodiments, the formulations of the application further comprise one or more pH adjusters, such as acidic, basic, or buffering components. These components may be added to provide the optimal pH balance for the skin. They may also be added to provide an optimal pH for one or more the components of the formulation. In some embodiments the pH of the formulations is adjusted to about 6 to about 7.5.

In some embodiments, the pH adjuster is selected from sodium hydroxide and potassium citrate. In some embodiment, the one or more pH adjusters are present in the formulation in an amount of about 0.05% wt % to about 2.0% wt, about 0.1 wt % to about 1.0 wt %, or about 0.8 wt % to about 0.8 wt %.

In some embodiments, the one or more pH adjusters are in the aqueous phase or the external phase.

(b) Chelating Agents

In some embodiments, the formulations of the application further comprise one or more chelating agents. In some embodiments, the chelating agents bind to metals which can inhibit the activity of the antimicrobial preservatives. In some embodiments, the chelating agent is sodium phytate or ethylendiamine tetraacetic acid (EDTA). In some embodiments, the one or more chelating agents are present in the formulation in an amount of about 0.01% wt % to about 0.2% wt, about 0.02 wt % to about 0.1 wt %, or about 0.03 wt % to about 0.05 wt %.

In some embodiments, the one or more chelating agents are in the aqueous phase or the external phase.

(c) Humectants

In some embodiments, the formulations of the present application further include one or more humectants. In some embodiments, the one or more humectants include, but are not limited to, glycerine (which also acts as an additional solvent).

In some embodiments, the one or more humectants are present in the formulation in an amount of about 0.5 wt % to about 10% wt, about 1 wt % to about 7 wt %, or about 2 wt % to about 5 wt %.

In some embodiments, the one or more humectants are in the aqueous phase.

(d) Solubilizers

In some embodiments, the formulations of the present application further include one or more solubilizers. In some embodiments, the one or more solubilizers include, but are not limited to, inulin lauryl carbonate.

In some embodiments, the one or more solubilizers are present in the formulation in an amount of about 0.01 wt % to about 5% wt.

In some embodiments, the one or more solubilizers are in the external phase.

(f) Further Active Ingredients

In some embodiments, the transdermal formulation of the present application further comprises other active ingredients. As used herein, active ingredients may include active molecules derived from natural, synthetic or semi-synthetic means, as well as other active ingredients.

In some embodiments, the further active ingredient is solubilized or dispersed in an effective amount of a suitable vehicle (e.g. solvent(s) or diluent(s)). A skilled person can readily determine which solvents or diluents will be appropriate for a particular API.

In some embodiments, the further API is included in an amount of about 0.01 wt % to about 1 wt %.

(g) Penetration Enhancer

In some embodiments the transdermal formulation of the present application further comprises penetration enhancers known in the art, for example, ethoxydiglycol (transcutanol), dimethyl isosorbide and mixtures thereof.

In some embodiments, the penetration enhancer is present in the formulation in an amount of about 1 wt % to about 30 wt % or about 3 wt % to about 20 wt %.

In some embodiments, the penetration enhancers are combined with one or more Sea Buckthorn extract in the external phase.

(h) Thickening Agents

In some embodiments, the transdermal formulation of the present application further comprises one or more thickening agents. In some embodiments, the thickening agents are present in the external phase and, in some embodiments the thickening agents enhance the stability of the formulation. In some embodiments, the thickening agents are one or more natural or synthetic polymers, such as, polyacrylates, polyisobutenes and polysorbates and mixtures thereof.

In some embodiments, the thickening agents are included in the external phase in an amount of about 0.01 wt % to about 5 wt % or about 0.5 wt % to about 2.0 wt %.

In some embodiments, the formulations of the present application are prepared using a process that comprises:

a) heating an aqueous phase comprising water and at least one water soluble emulsion stabilizer to a first temperature;
(b) heating an oil phase comprising at least one emulsifier, at least one oil soluble emulsion stabilizer, at least one emollient comprising at least one flavonoid, and at least one other emollient to the first temperature;
(c) adding the aqueous phase to the oil phase with stirring at the first temperature and continuing to stir at the first temperature until an emulsion is formed;
(d) cooling the emulsion in (c) to a second temperature; and, in any order:
(e) adding one or more external phases comprising at least one phospholipid-complexed flavonoid and celecoxib to the emulsion at the second temperature; and
(f) adding one or more preservative phases to the emulsion.

In some embodiments, the first temperature is about 65° C. to about 85° C., about 70° C. to about 80° C., or about 75° C.

In some embodiments, the second temperature is about 30° C. to about 50° C., about 35° C. to about 45° C., or about 40° C.

In some embodiments, penetration enhancers are used to dissolve celecoxib in the external phase.

In some embodiments, the process further comprises preparing the external phase wherein at least one flavonoid-extract is added.

In some embodiments, the process further comprises preparing the external phase wherein the at least one phospholipid-complexed flavonoid is stirred with water for a sufficient amount of time to become hydrated prior to being combined with the remaining ingredients for the external phase.

In some embodiments, the phases and emulsions are mixed with a homogenizer prior to combining with other phases.

In some embodiments of the application the formulations described herein are in the form of a cream, gel, liquid suspension, ointment, solution, patch or any other form for transdermal administration and the contents of the formulation adjusted accordingly. In some embodiments, the formulations are in the form of a cream. In some embodiments the cream has a viscosity of about 50000 cps to about 400000 cps, or about 70000 cps to about 350000 cps as measured using a Brookfield RVT T4-2.5, T4-3.0 or T4-4.0 RPM instrument at room temperature.

In some embodiments of the application, the formulation maintains its initial color, pH and/or viscosity for at least one month, at least two months or at least three months.

### III. Methods of the Application

In some embodiments, the present application includes a method for transdermal administration of celecoxib comprising administering an effective amount of one or more of the formulations of the present application to a subject in need thereof. In further embodiments, the present application includes a use of one or more formulations of the present application for the administration of celecoxib to a subject.

The present application includes therapeutic methods and uses of the formulations described herein. In some embodiments, the formulations are used in methods to treat one or more celecoxib-responsive diseases and conditions.

Accordingly, the present application includes methods for treating one or more celecoxib-responsive diseases and conditions, comprising administering an effective amount of a transdermal formulation of the application to a subject in need thereof. Also included is a use of a transdermal formulation of the application to treat one or more celecoxib-responsive diseases and conditions. In some embodiments the celecoxib-responsive disease and condition is selected from one or more of acute pain, chronic pain, nociceptive pain, neuropathic pain, inflammation and cancer. In some embodiments, the transdermal formulations of the application are to treat acute pain selected from musculoskeletal pain, postoperative pain and surgical pain. In some embodiments, the transdermal formulations of the application are to treat chronic pain selected from rheumatoid arthritis, osteoarthritis, ankylosing spondylitis, pain associated with cancer and fibromyalgia.

In some embodiments, the formulations of the application are used in conjunction with other therapies to treat celecoxib-responsive diseases and conditions.

### Examples

The following non-limiting examples are illustrative of the present application:

### Example 1: Preparation of Exemplary Transdermal Base Formulations Comprising Celecoxib

Topical formulations comprising celecoxib were prepared using the ingredients listed in Tables 1, 3, 5, and 7.

### Procedure for Making Formulations 1 and 4

Step A: In a stainless steel container, the ingredients of Phase A were combined and heated to 75° C.

Step B: In the main tank, ingredients of Phase B were combined, ensuring the thickening agent was well dispersed. Once a homogenous solution was achieved, the solution mixture from Step A was added into the main tank, followed by rapid stirring until complete emulsification, about 2-3 minutes. The solution mixture in the main tank was gradually cooled to a reaction temperature of 35-40° C., while stirring.

Step C: In a stainless steel container, ingredients of Phase C were combined.

Step D: In a stainless steel container, ingredients of Phase D were combined.

Step E: In a stainless steel container, ingredients of Phase E were combined.

Step F: In a stainless steel container, ingredients of Phase F were combined.

Step G: In a stainless steel container, ingredients of Phase G were combined.

Step H: While stirring, mixtures from steps C-G were added to the mixture from step B. The combined solution mixtures were stirred until homogenous and then cooled to room temperature.

### Procedure for Making Formulation 2

Step A: In a stainless steel container, the ingredients of Phase A were combined and heated to 75° C.

Step B: In the main tank, ingredients of Phase B were combined, ensuring the thickening agent was well dispersed. Once a homogenous solution was achieved, the solution mixture from Step A was added into the main tank, followed by rapid stirring until complete emulsification, about 2-3 minutes. The solution mixture in the main tank was gradually cooled to a reaction temperature of 35-40° C., while stirring.

Step C: In a stainless steel container, ingredients of Phase C were combined.

Step D: In a stainless steel container, ingredients of Phase D were combined.

Step E: In a stainless steel container, ingredients of Phase E were combined.

Step F: In a stainless steel container, ingredients of Phase F were combined.

Step G: While stirring, mixtures from steps C-F were added to the mixture from step B. The combined solution mixtures were stirred until homogenous and then cooled to room temperature.

### Procedure for Making Formulation 3

Steps A: In a stainless steel container, ingredients of Phase B were combined.

Step B: While stirring, mixture from step A was combined with exemplary base formulation 5. The combined solution mixtures were stirred until homogenous and then cooled to room temperature.

### Storage Stability of Formulation 1.

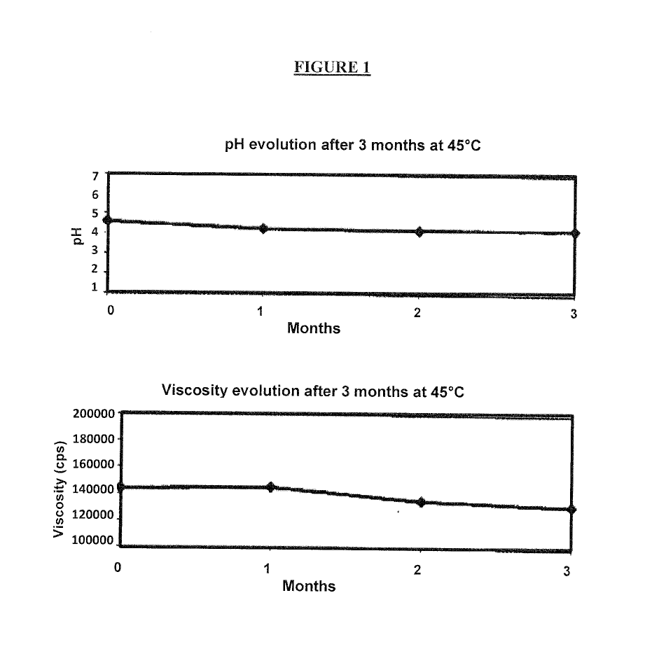

Formulation 1 was evaluated for its stability using four parameter measurements which included pH, texture, color and odour at 45° C. Formulation 1 maintained its stability in all four parameters over the course of 3 months providing an average pH evolution of 4.34±0.181 with a viscosity evolution averaging at 139100 cps±6401 as illustrated in FIG. 1. Furthermore, the cream produced a beige color. All measured parameters are illustrated in Table 2.

### Storage Stability of Formulation 2.

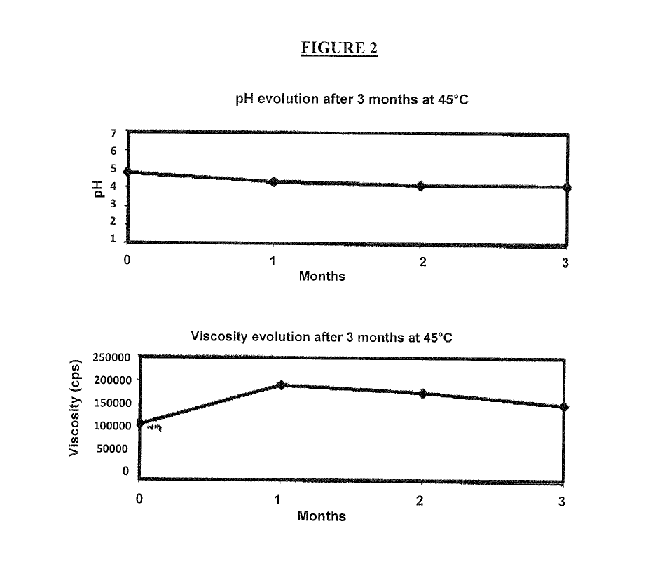

Formulation 2 was evaluated for its stability using four parameter measurements which included pH, texture, color and odour over a period of 3 months at 45° C. Formulation 2 maintained its stability in all four parameters over the course of 3 months providing an average pH evolution of 4.39±0.260 with a viscosity evolution averaging at 160375 cps±35234 as illustrated in FIG. 2. Furthermore, the cream produced a light beige color. All measured parameters are illustrated in Table 4.

### Storage Stability of Formulation 3.

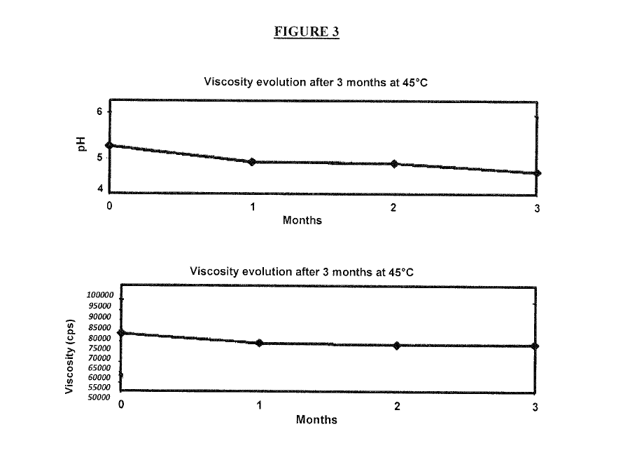

Formulation 3 was evaluated for its stability using four parameter measurements which included pH, texture, color and odour over a period of 3 months at 45° C. Formulation 3 maintained its stability in all four parameters over the course of 3 months providing an average pH evolution of 4.98±0.205 with a viscosity evolution averaging at 80525 cps±2136 as illustrated in FIG. 3. Furthermore, the cream produced a beige color. All measured parameters are illustrated in Table 6.

### Storage Stability of Formulation 4.

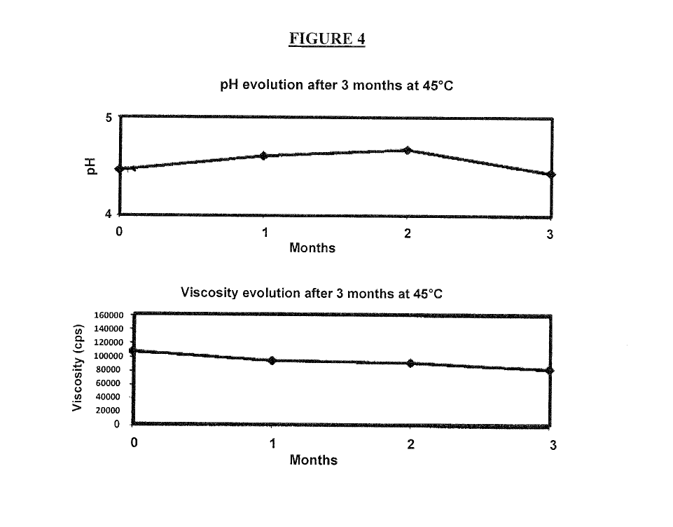

Formulation 4 was evaluated for its stability using four parameter measurements which included pH, texture, color and odour over a period of 3 months at 45° C. Formulation 4 maintained its stability in all four parameters over the course of 3 months providing an average pH evolution of 4.54±0.11 with a viscosity evolution averaging at 94700 cps±9855 as illustrated in FIG. 4. Furthermore, the cream produced a beige-yellow color after the first month, and became a beige color in months 2 and 3 and then turned to a dark beige color in the third month. All measured parameters are illustrated in Table 8.

### Example 2: Oral and Transdermal Celecoxib in Canine Sera and Synovial Fluid Methods

Celecoxib Protocol:

A Latin Square design was used employing six aged dogs with radiographic evidence of bilateral shoulder osteoarthritis. The treatment cycle consisted of six days twice daily followed by a period of seven days devoid of treatment. Blood and synovial fluid samples (from both stifles and shoulders) were collected on the last (sixth) day of drug application and on the last (seventh) day of the washout period.

Test article: 3% celecoxib in exemplary transdermal base formulation 4; ˜0.5 g gel per shoulder (2) and stifle joint (2) twice daily (i.e. 60 mg celecoxib total twice daily).

Control article: Exemplary transdermal base formulation 5; ˜1 g gel per shoulder joint twice daily.

Positive control: 30 mg celecoxib in capsule PO twice daily.

Serum and Synovial Fluid Collection:

Whole blood and synovial fluid samples were collected from the dogs 60 mins post treatment on the 7^{th} day, and additional samples were collected after a 7 day washout. Baseline synovial fluid was collected from untreated canine subjects. Synovial fluid was collected under sedation (medetomidine, 0.01 mg/kg) and butorphenol (0.1 mg/kg, IV). The fluid was collected by arthrocentesis into the joint following surgical preparation of the area. Synovial fluid was collected from the stifle or shoulder joints.

Analytical Method Development and Quantification:

The method for quantification of celecoxib in serum, plasma and synovial fluid was completed by the National Research Council of Canada (NRC). The results were categorized and summarized for average and deviations for each treatment group and time period.

Results & Discussion

The objective of this study was to determine if the transdermal formulations of the application were capable of delivering celecoxib either into the systemic circulation and joint synovial fluid and compare transdermal versus oral routes of administration. A canine model was used over two time periods. The first time period consisting of a twice daily drug administration regimen for six days to assess concentrations predictive of chronic use. The second time period followed immediately after, wherein all animals were withdrawn of drug administration and tested after seven days to determine if celecoxib was completely metabolized.

The results of this study include several observations regarding the relative bioaccumulation of celecoxib in the systemic circulation when comparing oral versus transdermal routes of administration.

Table 10A-C illustrate the drug concentrations used during the chronic administration period of six days (A), the total number of animals with biological samples (analyzed in duplicate) for each drug treatment and time period (B), and the final drug concentrations (analyzed in duplicate) for each drug treatment and time period (C).

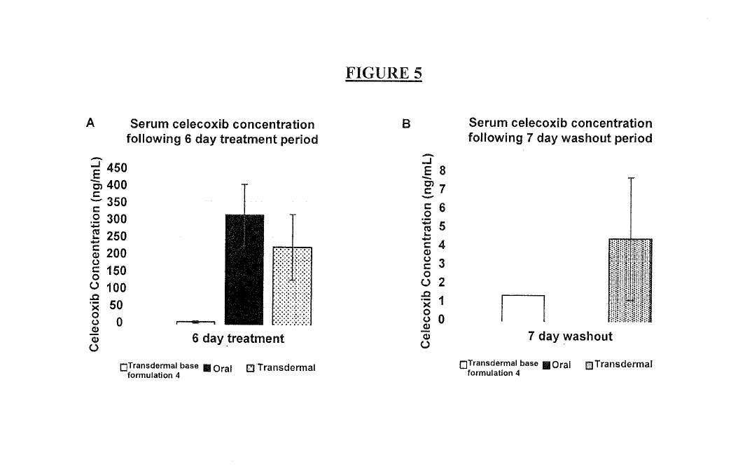

The chronic (6 day) administration of oral or transdermal celecoxib yielded a serum drug concentration of 312 ng/mL and 221 ng/mL, respectively (FIG. 5A). This data indicates equivalent serum drug levels are achieved regardless of route of administration. Further, following a seven day period devoid of drug administration, the levels of celecoxib in oral or transdermal treatments decreased to zero and 4 ng/mL, respectively (FIG. 5B). This data indicates that a one week washout period is sufficient to reduce circulating drug concentrations by >95%.

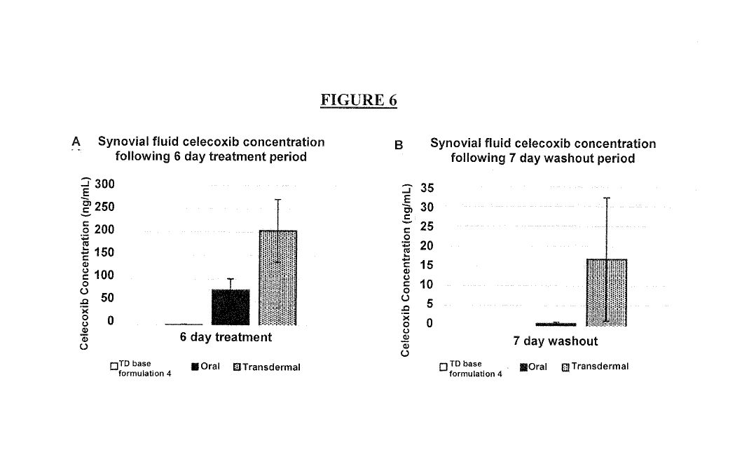

Similarly, a chronic (6 day) administration of oral or transdermal celecoxib yielded a synovial fluid drug concentration of 76 ng/mL and 203 ng/mL, respectively (FIG. 6A). Although a trend toward higher synovial levels using transdermal application is present, the high variability of drug levels abrogates a statistically significant result (p value=0.1). Following a seven day period devoid of drug administration, the levels of synovial celecoxib in oral or transdermal treatments decreased to 1 ng/mL and 17 ng/mL, respectively (FIG. 6B). This data indicates that a one week washout period is sufficient to reduce circulating drug concentration by >90%. Similar to the chronic administration results, although a trend towards higher synovial levels using transdermal application is present, the high variability of drug level abrogates a statistically significant result (p value=0.4).

The oral and transdermal regimens did not include the use of equimolar exposures, particularly transdermal treatment equated to two-fold the exposure concentration of the oral group. Given that the levels of celecoxib during chronic treatment are roughly equivalent for both oral and transdermal administration and that the exposure levels are unequal, the transdermal routes require twofold the amount of drug to achieve the same circulating concentration. Although the synovial fluid levels of celecoxib during chronic treatment and following the seven day washout period are numerically higher than the oral route, the results are not statistically significant, therefore evidence of a distinct tissue distribution of celecoxib from transdermal treatment were not determined.

### Example 3: Comparison of Oral and Transdermal Anti-Inflammatories on Joint Inflammation Using the Sodium Urate Induced Synovitis Model in Beagle Dogs

Study Design:

The study was a randomized, blinded, preclinical study using a controlled, parallel, matched-group design. Thirty-two subjects meeting the inclusion criteria were enrolled in the trial and allocated to 4 balanced groups; therefore one group received 2 treatments separated by a minimum of 14 days. Following induction of synovitis by sodium urate injections, effectiveness of transdermal formulations were evaluated using a pain questionnaire assessment and joint measurements at various time points.

Experimental Materials:

Test Article 1:

| | | |
|---|---|---|
| i) | Name | Transdermal Celebrex 3% (celecoxib) |
| (ii) | Dosage form | Transdermal cream |
| (iii) | Dose tested | 37.5 mg/kg |
| (iv) | Drug storage during study | refrigerated 2-4° C. |

Positive Control 1:

| | | |
|---|---|---|
| i) | Name | PLO Celebrex 3% |
| (ii) | Dosage form | Transdermal cream |
| (iii) | Dose tested | 37.5 mg/kg |
| (iv) | Drug storage during study | refrigerated 2-4° C. |

Positive Control 2:

| | | |
|---|---|---|
| i) | Name | Oral Celecoxib |
| (ii) | Dosage form | Powder in gelatin capsule |
| (iii) | Dose tested | 3 mg/kg |
| (iv) | Drug storage during study | refrigerated 2-4° C. |

Materials and Methods

(1) Test System

32 beagle dogs obtained from the Vivocore Inc. colony were included in the study. Ages of the dogs ranged from 1.0 to 11.6 years at study initiation. There were 17 males and 15 females included.

(2) Selection and Allocation of Animals

Animals in good health as determined by historical health records were included in the study. Included subjects had no clinical signs of osteoarthritis and a baseline cumulative pain score of 5 or less determined using a validated pain questionnaire. Subjects were divided into two groups based on sex. The dogs were then divided into four groups such that groups were balanced for age. The four groups were then randomly assigned to treatment conditions.

(3) Acclimation and Pre-treatment of Test System

All animals involved in this investigation were housed at the Vivicore facility for no less than six months.

(4) Administration of Test Article

On days 0, 2, 7 and 22, subjects who were schedules for synovitis induction were treated according to their assigned treatment condition. Test article administration occurred 30 minutes following synovitis induction, i.e. sodium urate injection. Transdermal treatment doses were measured in milligrams using an analytical scale and were then applied directly to the induce stifle. Transdermal treatments were rubbed into the dog's skin for a minimum of 1 minute. Oral treatments were administered with attention to complete delivery and retention of the entire intended dose.

(5) Housing and Management of Test System

Housing: Dogs were individually housed in metabolic cages in compliance with the recommendations of the Canadian Council on Animal Care. All cages and housing areas were cleaned daily. Dogs were exercised according to standard operating procedure on non-testing days and following completion of pain and inflammation assessments on test days.

Environmental: Environmental management including lighting, ventilation, temperature and humidity regulation were maintained and controlled according to standard operating procedures as described below. A combination of commercially acceptable fluorescent lighting and natural light was provided for the dogs. Heating and cooling was electronically controlled and was set to maintain the animal room in a temperature range from 15° C. to 28° C. The housing room ventilation was designed to provide 18 filtered air changes per hour.

Feed and Water: All animals were fed according to standard operating procedure at the end of each day. Dogs were fed a standard commercial dry diet to maintain body condition. Food consumption was not recorded. Water was provided ad libitum.

(6) Procedures and Data Recorded

A) Twice daily observations: Animals were observed twice daily over the course of the study according to facility standard operating procedures.

B) Animal body weights: Animal body weights were determined (in kg) with a certified, verified scale on study days −1 and 21. The recorded weights were used to determine individual treatment doses.

C) Anesthesia and sodium urate injection: Induction of synovitis by sodium urate injection occurred on days 0, 2, 7 and 22, as outlined above. Anesthesia was induced with propofol (8 mg/kg W to effect). Subjects were then intubated and anesthesia was maintained with an isoflurane-oxygen mixture. If IV administration of the propofol was not possible due to inaccessibility of the vein, the subjects were masked down using the isoflurane-oxygen mixture and the event was recorded on the anesthesia record.

Prior to injection, sodium urate crystals were mixed with sterile saline to produce a solution with a concentration of 20 mg/mL. The solution was sonicated for 60 minutes and the pH was adjusted to a suitable level for injection (between 6.9 and 7.2) with the addition of either hydrochloric acid or sodium hydroxide.

Following aseptic surgical preparation of the injection area, the joint was aspirated and the presence of synovial fluid confirmed accurate location for injection. For each animal, 1 ml of the sodium urate solution was injected into either the right or left stifle. For the second urate injection (if applicable), the stifle contralateral to that used for the first urate injection was used. Each treatment group had the same number of dogs induced in the left stile and right stifle.

D) Pain questionnaire assessment: The pain questionnaire is a laboratory adaption of the validated canine brief pain inventory (CBPI) questionnaire, which is a clinical questionnaire used to evaluate pain level based on owner responses. Modifications to the questionnaire account for differences between owner pain evaluation of pets and pain evaluation of laboratory dogs by technical staff. Specifically, the ability for each dog to walk, trot, gallop, rear, jump over a low obstacle, climb and descend stairs, jump down from a perch and general activity is evaluated in parallel to subjective evaluation of pain observed during each behavior. Assessments were performed by the same trained technician across the study.

All subjects underwent pain questionnaire pre-screening within 3 days of each scheduled induction. Cumulative pain scores were used to confirm inclusion criteria and also served as the baseline scores for the treatment administered. Subjects assigned to more than one treatment group were re-screened prior to the second induction. This served two purposes; first to ensure animals returned to normal pain levels following the initial sodium urate injection, and second, to ensure that subject's cumulative pain scores continued to be below 5 as per the inclusion criteria for the study.

On days sodium urate was injected, animals were assessed on the questionnaire at 4.5, 8.5, 24.5 and 30.5 hours following induction of synovitis (±15 minutes). At these time points, the pain questionnaire was performed immediately following measurement of the patella/patellar tendon (knee cap) width as described below.

E) Caliper measurements: Within thirty minutes of synovitis induction and 4.5, 8.5, 24.5 and 30.5 hours following induction (±15 minutes), the width of the patella and the patellar tendon were measured in millimeters using Venier 150 mm plastic gauge calipers which measured in increments of 1 mm. Measurements of both the left and right stifle were performed by the same technician who took three separate caliper measurements of each joint. The three measurements from each joint were averaged. On the day prior to the pre-induction caliper measurement, the hair of the measurement areas were clipped and marked to facilitate consistent measures.

F) Blinding the study: The treatment given to each animal was not revealed to technicians collecting the pain and caliper data. The study was blinded to all personnel in the investigation with the exception of the persons involved with preparing and administering the investigational products, the person responsible for performing the allocation, the study coordinator and scientific director. Non-blinded personnel did not collect data other than at the time of treatment. Treatment condition information was kept in a locked archive room over the course of the study.

Results and Discussion

The objective of this study was to evaluate the effects of celecoxib on measures of pain and inflammation in a canine model of inflammatory synovitis induced by sodium urate injection. Celecoxib was administered either transdermally in a transdermal base formulation of the application, or transdermally in PLO or oral dosing.

To assess the effect of the transdermal formulations of the application comprising celecoxib compared to the positive and negative controls, statistical analyses were conducted. The cumulative pain scored and stifle measurements were analyzed using a repeated-measures analysis of variance (ANOVA) with the treatment group (7 factors( ) as between-subject measure and time-point (5 factors for pain and 4 factors for the inflammation measure) as a within-subject measure. Post-hoc Fisher's test was used to examine differences as appropriate. The Statistica 11 (Statsoft, Tulsa, Okla.) statistical software package was used with significance set to p=0.5.

Celebrex

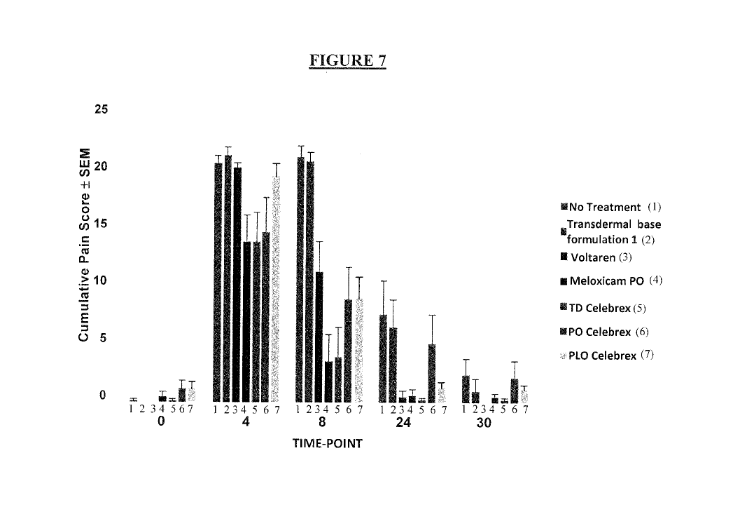

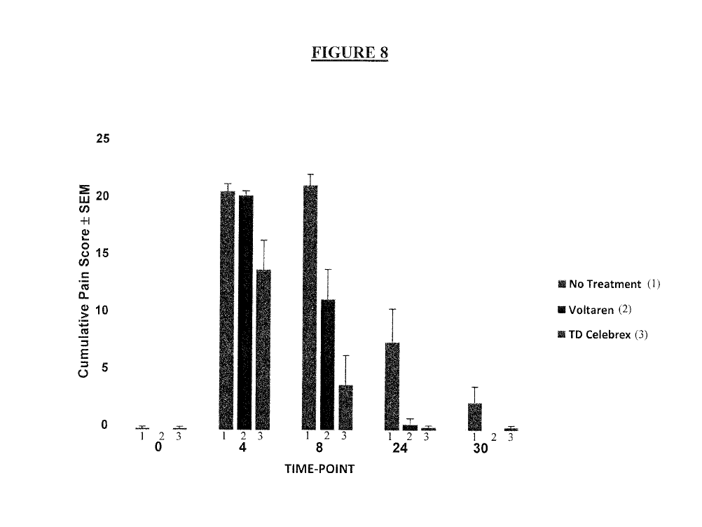

The cumulative pain score analysis revealed significant effects of both treatment group [F(6,49)=8.06; p=0.00004] and time-point [F(4,196)=213.78; p<0.00001] as well as a treatment group by time-point interaction [F(24,196)=5.44; p<0.00001]. In comparison to no treatment, pain was significantly reduced by Celebrex at the 4 time-point (p=0.01) and 8 time-point (p<0.01) hours after oral administration, 4(p<0.01), 8(p<0.01) and 24(p<0.01) hours after delivery in the exemplary transdermal formulations of the application (TD Celebrex). By contrast, Voltaren and Celebrex delivered in PLO significantly reduced pain at 8 and 24 hours after treatment ((p<0.05 in all cases). The positive control, oral meloxicam reduced pain compared to no treatment at 8, 24 and 30 hours (p<0.01 in all cases). Moreover, Celebrex delivered in exemplary formulation 1 (TD Celebrex) and meloxicam were significantly superior to oral delivery of Celebrex at 8 and 24 hours (p<0.05 in all cases; FIG. 7 and FIG. 8). At the earliest time point (4 hours), exemplary transdermal formulation 1 and oral Celebrex resulted in similar pain scores, however the effects of the exemplary transdermal formulation 1 (TD Celebrex) persisted through the later time points.

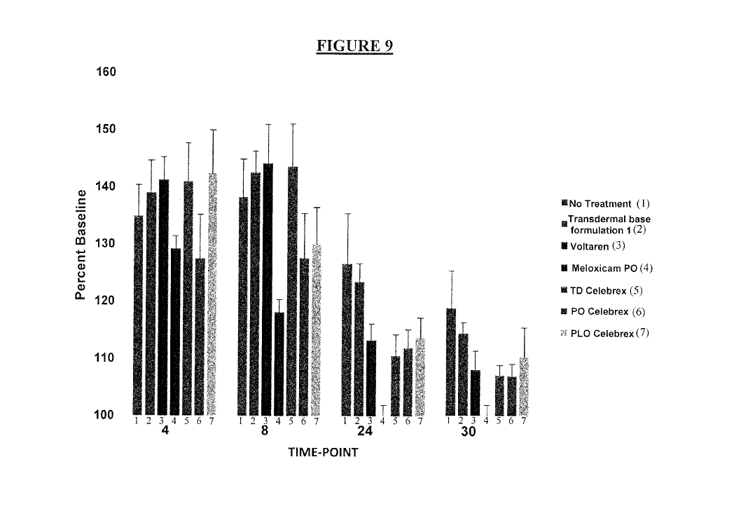

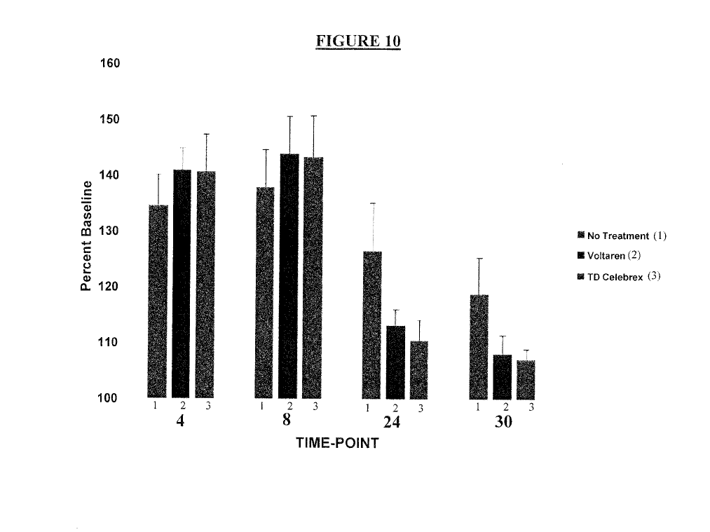

The percent baseline measure of inflammation analysis revealed significant effects of treatment group [F(6,49)=2.51; p<0.034] and time-point [F(3,147)=99.77; p<0.00001] as well as a treatment group by time-point interaction [F(18,147)=1.95; p=0.016]. In comparison to no treatment, inflammation was significantly reduced only by Celebrex delivered in exemplary formulation 1 at the 24 hour (p=0.03) and orally (p+0.05), although trends for the other treatments were also evident at the same time-point (FIGS. 9 and 10). The positive control meloxicam administered orally reduced inflammation at 8, 24 and 30 hours compared to no treatment. Oral Celebrex (p=0.03) and meloxicam (p<0.01) significantly reduced inflammation compared to Voltaren at 8 hours, but no differences between Voltaren and transdermal (TD) celebrex in exemplary formulation 1 were found.

The current study demonstrated that Celebrex formulation in exemplary transdermal formulation 1 compared to negative control, significantly decreased cumulative group pain scores at 4, 8 and 24 hours post treatment in this sodium urate synovitis model in dogs and was superior to the oral and PLO delivery method. Moreover, transdermal formulation 1 significantly reduced inflammation at 24 hours compared to negative control; no effects on inflammation were seen in the Voltaren and oral Celebrex and PLO groups. Compared to Voltaren, pain was significantly lowered by Celebrex administered in transdermal formulation 1 at the 4 and 8 hour post-dose time-points.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
Formulation 1

| | Ingredients | % |
|---|---|---|
| Phase A | Emulsifier | 4.0% |
| | Wax Stabilizer | 3.0% |
| | Wax Emollient | 1.0% |
| | Polar Emollient Oils | 8.0% |
| | Medium Polar Emollient | 2.0% |
| Phase B | Water | 50.5% |
| | Humectant | 4.0% |
| | Thickening Agent | 0.40% |
| Phase C | Preservatives | 1.0% |
| Phase D | Preservative | 0.10% |
| | Flavanoid-containing | 6.0% |
| | Extracts | |
| | Thickening Agent | 1.0% |
| Phase E | Water | 3.0% |
| | Phospholipid-complexed | 2.0% |
| | Flavanoid | |
| Phase F | Celecoxib | 3.0% |
| | Penetration Enhancer | 6.0% |
| Phase G | Water | 5.0% |
| | Total | 100.00% |

TABLE 2
Stability parameters, pH, texture, color and odour for formulation 1 at
45° C. for 90 days

| | | | Viscosity | | | |
|---|---|---|---|---|---|---|
| | Avg. | | (T4-2.5 | | | |
| | Temp | | rpm, | | | |
| Months | (° C.) | pH | cps) | Appearance | Colour | Scent |
| 0 | 25 | 4.61 | 144000 | cream | beige | characteristic |
| 1 | 25 | 4.27 | 145000 | cream | beige | characteristic |
| 2 | 25 | 4.22 | 135400 | cream | beige | characteristic |
| 3 | 25 | 4.26 | 132000 | cream | beige | characteristic |

TABLE 3
Formulation 2

| | Ingredients | % |
|---|---|---|
| Phase A | Emulsifier | 4.0% |
| | Wax Stabilizer | 3.0% |
| | Wax Emollient | 1.0% |
| | Polar Emollient Oils | 8.0% |
| | Medium Polar Emollient | 2.0% |
| Phase B | Water | 40.5% |
| | Humectant | 4.0% |
| | Thickening Agent | 0.40% |
| Phase C | Preservative | 1.0% |
| Phase D | Preservative | 0.10% |
| | Flavanoid-containing | 6.0% |
| | Extracts | |
| | Thickening Agent | 1.0% |
| Phase E | Water | 3.0% |
| | Phospholipid-complexed | 2.0% |
| | Flavanoid | |
| Phase F | Celecoxib | 8.0% |
| | Penetration Enhancer | 16.0% |
| | Total | 100.00% |

TABLE 4
Stability parameters, pH, texture, color and odour for formulation 2 at
45° C. for 90 days

| | | | Viscosity | | | |
|---|---|---|---|---|---|---|
| | Avg. | | (T4-2.5 | | | |
| | Temp | | rpm, | | | |
| Months | (° C.) | pH | cps) | Appearance | Colour | Scent |
| 0 | 25 | 4.77 | 112300 | cream | light | characteristic |
| | | | | | beige | |
| 1 | 25 | 4.33 | 192700 | cream | light | characteristic |
| | | | | | beige | |
| 2 | 25 | 4.20 | 179300 | cream | light | characteristic |
| | | | | | beige | |
| 3 | 25 | 4.25 | 157200 | cream | Light | characteristic |
| | | | | | beige | |

TABLE 5
Formulation 3

| | Ingredients | % |
|---|---|---|
| Phase A | Transdermal base | 80.0% |
| | formulation | |
| Phase B | Celecoxib | 8.0% |
| | Penetration Enhancer | 12.0% |
| | Total | 100.00% |

TABLE 6
Stability parameters, pH, texture, color and odour for formulation 3 at
45° C. for 90 days

| | | | Viscosity | | | |
|---|---|---|---|---|---|---|
| | Avg. | | (T4-4.0 | | | |
| | Temp | | rpm, | | | |
| Months | (° C.) | pH | cps) | Appearance | Colour | Scent |
| 0 | 25 | 5.27 | 83700 | cream | beige | characteristic |
| 1 | 25 | 4.95 | 79500 | cream | beige | characteristic |
| 2 | 25 | 4.94 | 79100 | cream | beige | characteristic |
| 3 | 25 | 4.78 | 79800 | cream | beige | characteristic |

TABLE 7
Formulation 4

| | Ingredients | % |
|---|---|---|
| Phase A | Emulsifier | 4.0% |
| | Wax Stabilizer | 3.0% |
| | Wax Emollient | 1.0% |
| | Polar Emollient Oils | 8.0% |
| | Medium Polar Emollient | 2.0% |
| Phase B | Water | 50.5% |
| | Humectant | 4.0% |
| | Thickening Agent | 0.20% |
| Phase C | Preservatives | 1.3% |
| Phase D | Flavanoid-containing | 6.0% |
| | Extracts | |
| | Thickening Agent | 1.0% |
| Phase E | Water | 3.0% |
| | Phospholipid-complexed | 2.0% |
| | Flavanoid | |
| Phase F | Celecoxib | 3.0% |
| | Water | 5.0% |
| Phase G | Penetration Enhancer | 6.0% |
| | Total | 100.00% |

TABLE 8
Stability parameters, pH, texture, color and odour for formulation 4 at
45° C. for 90 days

| | | | Viscosity | | | |
|---|---|---|---|---|---|---|
| | Avg. | | (T4-3.0 | | | |
| | Temp | | rpm, | | | |
| Months | (° C.) | pH | cps) | Appearance | Colour | Scent |
| 0 | 25 | 4.46 | 107800 | cream | beige | characteristic |
| | | | | | yellow | |
| 1 | 25 | 4.60 | 94600 | cream | beige | characteristic |
| 2 | 25 | 4.67 | 92400 | cream | beige | characteristic |
| 3 | 25 | 4.44 | 84000 | cream + | dark | characteristic |
| | | | | oil drop | beige | |

TABLE 9
Formulation 5

| | Ingredients | % |
|---|---|---|
| Phase A | Emulsifier | 4.0% |
| | Wax Stabilizer | 3.0% |
| | Wax Emollient | 2.0% |
| | Polar Emollient Oils | 10.0% |
| | Medium Polar Emollient | 3.0% |
| Phase B | Water | 58.70% |
| | Thickening agent | 0.5% |
| | Humectant | 4.0% |
| Phase C | Preservatives | 1.3% |
| Phase D | Flavonoid-containing | 7.0% |
| | extracts | |
| | Penetration Enhancer | 1.5% |
| | Thickening Agent | 1.0% |
| | Water | 2.0% |
| | Phospholipid-Complexed | 2.0% |
| | Flavonoid | |
| | Total | 100.00% |

TABLE 10
Outline of experimental design and drug concentration of celecoxib

Chronic administration (6 days)

| | Oral | Transdermal |
|---|---|---|
| (A) | (mg/day) | (mg/day) |
| Celecoxib | 60 | 120 |

| | Chronic administration (6 days) | | Washout (7 days) | |
|---|---|---|---|---|
| | Oral | Transdermal | Oral | Transdermal |
| (B) | (N) | (N) | (N) | (N) |
| Serum | 6 | 6 | 4 | 4 |
| Synovial fluid | 6 | 6 | 4 | 5 |

| | Chronic administration (6 days) | | Washout (7 days) | |
|---|---|---|---|---|
| | Oral | Transdermal | Oral | Transdermal |
| (C) | (ng/mL) | (ng/mL) | (ng/mL | (ng/mL) |
| Serum | 312 (±214) | 221 (±227) | 0 | 4 (±6) |
| Synovial | 76 (±57) | 203 (±165) | 1 (±1) | 17 (±35) |
| fluid | | | | |

Drug concentrations (C) described and include values for the standard deviation for each in brackets.
Statistically significant differences between oral and transdermal drug concentrations are highlighted ( *, Pvalue = 0.00002; ⁺ Pvalue = 0.02)

### REFERENCES

1. Frampton, J. E. (2007). A review of its use in the management of arthritis and acute pain. Drugs, 67, 2433-2474.
2. Prausnitz, M. R and Langer, R. (2008) Transdermal drug delivery. Nat.

Biotechnol, 26, 1261-1268.

1. A transdermal formulation comprising,
(a) an aqueous phase comprising water and at least one water soluble emulsion stabilizer;
(b) an oil phase comprising at least one emulsifier, at least one oil soluble emulsion stabilizer, at least one emollient comprising at least one flavonoid and at least one other emollient;
wherein the oil and aqueous phase form an emulsion;
(c) an external phase comprising at least one phospholipid-complexed flavonoid and celecoxib; and
(d) at least one preservative phase.

2. The transdermal formulation of claim 1, wherein celecoxib is present in the formulation in an amount of about 0.1 wt % to about 15 wt %, about 1 wt % to about 10 wt % or about 2 wt % to about 8 wt %.

3. The transdermal formulation of claim 1 further comprising at least one flavonoid-containing extract in the external phase.

4. The transdermal formulation of claim 1, in the form of a cream, gel, liquid suspension, ointment, solution or patch.

5. The transdermal formulation of claim 1, in the form of a cream.

6. The transdermal formulation of claim 5, wherein the cream has a viscosity of about 50000 cps to about 400000 cps, or about 70000 cps to about 350000 cps as measured using a Brookfield RVT T4-2.5, T4-3.0, or T4-4.0 RPM instrument at room temperature.

7. A method for the transdermal administration of celecoxib comprising administering an effective amount of one or more of the formulations of claim 1 to a subject in need thereof.

8. A method for treating a celecoxib-responsive disease or condition comprising administering an effective amount of one or more of the transdermal formulations of claim 1 to a subject in need thereof.

9. The method of claim 8, wherein the celecoxib-responsive disease or condition is selected from one or more of acute pain, chronic pain, nociceptive pain, neuropathic pain, inflammation and cancer.

10. The method of claim 9, wherein the acute pain is selected from musculoskeletal pain, postoperative pain and surgical pain.

11. The method of claim 9, wherein the chronic pain is selected from rheumatoid arthritis, osteoarthritis, ankylosing spondylitis, pain associated with cancer and fibromyalgia.

12. The transdermal formulation of claim 1, wherein the emulsifier in the oil phase is any oil-soluble fatty acid ester or mixture of fatty acid esters in which the fatty acid esters have a fatty acid composition similar to the fatty acid composition of skin.

13. The transdermal base formulation of claim 12, wherein the any oil-soluble fatty acid ester or mixture of fatty acid esters is a C₁₄-C₂₆-fatty acid esterified with a fatty acid alcohol selected from cetyl alcohol, cetaryl alcohol, lauryl alcohol, stearyl alcholol, myristyl alcohol and oleyl alcohol or with a sugar alcohol selected from sorbitol, glycerol, mannitol, inositol, xylitol, erythritol, threitol, arabitol and ribitol, or mixtures thereof.

14. The transdermal base formulation of claim 1, the oil phase emulsion stabilizer is one or more waxes.

15. The transdermal base formulation of claim 14, wherein the waxes are selected from animal and plant waxes and mixtures thereof.

16. The transdermal base formulation of claim 1, wherein the aqueous phase emulsion stabilizer is selected from natural polymers, gums and synthetic polymers, and mixtures thereof.

17. The transdermal base formulation of claim 1, wherein the one or more emollients comprising one or more flavonoid compounds are polar emollients selected from natural oils and extracts from plants.

18. The transdermal base formulation of claim 17, wherein the polar emollient is a natural oil or extract from citrus, Ginkgo biloba, tea, wine, cacao, onion, kale, parsley, red beans, broccoli, endive, celery, cranberries, blackberries, red raspberries, blackcurrants, acai, blueberries, bilberries, milk thistle, apples, hawthorn, Echinacea, grapes, and/or soy.

19. The transdermal base formulation of claim 1, wherein the at least one other emollient is selected from octyl palmitate, isopropyl stearate, isopropyl palmitate and octyl dodecanol.

20. The transdermal base formulation of claim 1, wherein the phospholipid in the phospholipid-complexed flavonoid is selected from a phosphatidylcholine, a phosphatidylethanolamine, phosphatidylinostinol and phosphatidylserine, and mixtures thereof, and the flavonoid is selected from quercetin, myrcetin, apigenin and rutin, and mixtures thereof.
